(19) United States
(12) Patent Application Publication
(10) Pub. No.: US 2023/0183736 A1
(43) Pub. Date: Jun. 15, 2023

(54) Use of insecticidal protein

(71) Applicant: BEIJING DABEINONG BIOTECHNOLOGY CO., LTD., Beijing (CN)

(72) Inventors: Tengyu CHANG, Beijing (CN); Qinyang WANG, Beijing (CN); Aihong ZHANG, Beijing (CN); Qing TAO, Beijing (CN)

(21) Appl. No.: 18/062,567

(22) Filed: Dec. 6, 2022

(30) Foreign Application Priority Data
Dec. 13, 2021 (CN) 202111515713.8

(51) Int. Cl. C12N 15/82; C12N 9/16

(52) U.S. Cl. CPC C12N 15/8286; C12N 9/16; C12Y 301/02001

(57) ABSTRACT

Related is a use of an insecticidal protein. The insecticidal protein may be used to control Apolygus lucorum. A method for controlling the Apolygus lucorum includes: allowing the Apolygus lucorum to be at least in contact with an ACh1 protein. In the present application, the ACh1 protein that can kill the Apolygus lucorum is produced in bacteria and/or a plant in vivo to control the Apolygus lucorum.

### CROSS-REFERENCE TO RELATED APPLICATION

The present application is based on and claims priority to Chinese application with a CN application number of 202111515713.8 and an application date of Dec. 13, 2021, the disclosure of which is hereby incorporated by reference again in its entirety.

### SEQUENCE LISTING

The instant application contains a Sequence Listing which has submitted electronically in ASCII format and is hereby incorporated by reference in its entirety. Said ASCII copy is named PN192851 SEQ LIST.xml and is 24,157 bytes in size. The sequence listing contains 14 sequences, which is identical in substance to the sequences disclosed in the CN application and includes no new matter.

### TECHNICAL FIELD

The present application relates to a use of an insecticidal protein, and in particular, to a use of an ACh1 protein for controlling damage of Apolygus lucorum to a plant by expressing in the plant.

### BACKGROUND

Apolygus lucorum is a paurometabola insect belonging to Hemiptera Miridae, which is one of the important pests in cotton production at present. Adults and nymphs of the Apolygus lucorum damage tender shoots, tender leaves and tender buds of cotton with piercing-sucking mouthparts. In the North, the pest occurs 3-5 generations a year; and in the South, the pest occurs 6-7 generations. The pest passes the winter by spawning in the dead branches of the cotton. When the temperature is higher than 10 degrees centigrade in the next spring, eggs hatch gradually; and then damage Astragalus smicus, alfalfa and the like, and then migrates in the cotton field to damage cotton. After the tender shoot is damaged, there are only “male” cotton with two hypertrophic cotyledons left; after the tender leaf is damaged, “broken leaf mania” with a large number of holes and crumpled leaves is formed; and the tender bud is withered and yellow and fallen off after being damaged. The Apolygus lucorum may damage the cotton in the whole growth period from germination to squaring, resulting in severe yield loss.

Cotton is an important commercial crop in China, but the economic loss caused by the Apolygus lucorum is huge every year. In order to control the Apolygus lucorum, main control methods usually used by people are agricultural control, chemical control and physical control.

The agricultural control is to comprehensively coordinate and manage an entire farmland ecosystem from multi-factors, and regulate crops, pests, and environmental factors so as to create a farmland ecological environment that facilitates crop growth but not facilitates the occurrence of the Apolygus lucorum. For example, before March, weeds from ridges, roadsides and graveyards are removed by combining with collected manure so as to eliminate overwintering eggs, so that the number of insect population in early spring can be reduced; green manure is harvested without residues; and when the green manure is turned over, the green manure is completely buried underground, so that the number of insects transferred to the cotton field can be reduced. By means of scientific and rational fertilization, the flourished growth of the cotton can be controlled, so as to alleviate the damage of plant bugs. However, agricultural control requires a lot of manpower and material resources, which is not easy to implement in the current production mode of combining planting and migration working in rural areas.

The chemical control is pesticide control, which uses chemical pesticides to kill pests and is an important part of the comprehensive management of the Apolygus lucorum. The chemical control has the characteristics of rapidity, convenience, simplicity and high economic benefits, and is an essential emergency measure, especially in the case of a large occurrence of the Apolygus lucorum. Currently, the chemical control method is mainly to apply organophosphorus and carbamate chemical pesticides. In addition, conventional pesticides such as organochlorines and pyrethroids still have high contact activity against the adults of the Apolygus lucorum. Wherein, 5 conventional pesticides of malathion, chlorpyrifos, bifenthrin, methomyl and endosulfan have desirable insecticidal effects. A novel pyrrole insecticide shows high virulence against the Apolygus lucorum, and an insect growth regulator and antibiotics and botanical insecticides do not have obvious contact activity against the Apolygus lucorum. In addition, hole application in the cotton field with imidacloprid granules and root application with liquid preparations are effective means for controlling the damage of the Apolygus lucorum. However, insecticides such as organophosphorus and organochlorine are not only toxic to other insects in the cotton fields, but also to other beneficial animals. In addition, due to long residence time, long-term pollution is caused to the soil and underground water, which does not facilitate the sustainable development of environments.

Physical control is mainly based on the response of pests to various physical factors in environmental conditions, using various physical factors such as light, electricity, color, temperature and humidity, as well as mechanical devices for trapping, radiation sterility and other methods to control pests. Although it is reported that the Apolygus lucorum has a certain tendency towards turquoise or blue, but in an entire turquoise environment of the cotton field, an actual attractive effect is not ideal.

In order to solve the limitations of the agricultural control, the chemical control and physical control in practical applications, it is found from researches by scientists that some insect-resistant transgenic plants may be obtained by transferring insect-resistant genes encoding insecticidal proteins into plants so as to control plant pests.

Pest-resistant crops have been developed by genetically engineering crops to introduce a Bacillus thuringiensis (Bt) protein into crops. For example, Cry1Ab is used to develop corns resistant to corn borer. At present, these genetically modified crops are widely used in agriculture and provide farmers with an environmentally friendly alternative to traditional insect control methods. Although the genetically modified crops have been shown to be quite effective against lepidopteran pests (the corn borer, bollworm, and the like), no genetically modified crops have been found that can control the Apolygus lucorum. The main reason for this is that no Cry protein has been found to be virulent to the Apolygus lucorum.

ACh1 is a new class of insecticidal proteins, which is completely different from the traditional Bt protein. By analyzing a protein secondary structure, the protein is speculated to belong to a β-pore forming protein. The mechanism of action of such proteins is generally enzymatic cleavage activation, binding with receptors, formation of oligomers, and pore-forming on membrane surfaces. The enzymatic cleavage activation in insect gut, receptor binding on the insect gut and a physicochemical environment in the insect gut determine whether the transmembrane pore can form in cell membranes of the insect gut. After such type of protein is secreted by the bacteria, it needs to be digested in a target body to form an active protein. The enzyme cleavage process is mainly performed at an amino-terminal or carboxyl-terminal of the protein, to turn the protein into an active fragment. The active fragment binds to a receptor on an epithelial cell membrane of the insect gut to form oligomer, and inserts into a gut membrane, so that a transmembrane pore appears on the cell membrane, and the osmotic pressure change and pH balance and the like inside and outside the cell membrane are destroyed, and the digestion process of the insects is disrupted, finally resulting in death of the insects.

The ACh1 protein has been reported to have inhibitory activity against silkworm and potato beetles. However, there is no report that the damage of the Apolygus lucorum may be controlled by producing a plant expressing the ACh1 protein so far.

### SUMMARY

The present application is intended to provide a use of an insecticidal protein, and for the first time provide a method for controlling Apolygus lucorum by producing a transgenic plant expressing an ACh1 protein, to effectively overcome technical defects in agricultural control, chemical control and physical control in the prior art.

In order to achieve the above objective, the present application provides a method for controlling Apolygus lucorum, including allowing the Apolygus lucorum to be at least in contact with an ACh1 protein.

Further, the ACh1 protein is present in a host cell that produces at least the ACh1 protein, and the Apolygus lucorum is in contact with at least the ACh1 protein by ingesting the host cell.

Further, the ACh1 protein is present in bacteria or a transgenic plant that produces at least the ACh1 protein, the Apolygus lucorum is in contact with at least the ACh1 protein by ingesting the bacterium or a tissue of the transgenic plant, and after contacting, the growth of the Apolygus lucorum is inhibited and/or death is caused, so as to achieve the control of the damage of the Apolygus lucorum to plants.

The transgenic plant may be in any growth stages.

The tissue of the transgenic plant is a bud, a leaf, a cotton boll, a tassel, an ear, or a filament.

The control of the damage of the Apolygus lucorum to the plants does not vary with planting location and/or planting time.

The plant is cotton, soybeans or rapes.

A step before the contacting step is to plant a plant containing polynucleotide encoding the ACh1 protein.

On the basis of the above technical solution, the ACh1 protein is an ACh1_1 protein, an ACh1_2 protein, an ACh1_3 protein, or an ACh1_4 protein.

Preferably, the ACh1 protein has an amino acid sequence shown in SEQ ID NO:1, SEQ ID NO:2, SEQ ID NO:3, or SEQ ID NO:4.

On the basis of the above technical solution, the plant further includes at least one second nucleotide different from nucleotide encoding the ACh1 protein.

Further, the second nucleotide encodes a Cry-like insecticidal protein, a Vip-like insecticidal protein, a protease inhibitor, lectin, α-amylase, or a peroxidase.

Preferably, the second nucleotide encodes a Cry3Bb protein.

Further, the Cry3Bb protein has an amino acid sequence shown in SEQ ID NO:9. The second nucleotide has a nucleotide sequence shown in SEQ ID NO:10.

Optionally, the second nucleotide is dsRNA that inhibits an important gene in a target insect pest.

In order to achieve the above objective, the present application further provides a use of an ACh1 protein for controlling Apolygus lucorum.

In order to achieve the above objective, the present application further provides a method for producing a plant for controlling Apolygus lucorum, including introducing a polynucleotide sequence encoding an ACh1 protein into a genome of the plant.

In order to achieve the above objective, the present application further provides a method for producing a plant seed for controlling Apolygus lucorum, including hybridizing a first plant obtained by the method with a second plant, so as to produce a seed containing a polynucleotide sequence encoding an ACM protein.

In order to achieve the above objective, the present application further provides a method for cultivating a plant for controlling Apolygus lucorum. The method includes the following operations.

At least one plant seed is planted, and a genome of the plant seed includes a polynucleotide sequence encoding an ACh1 protein.

The plant seed is grown into a plant.

The plant is grown under conditions that the Apolygus lucorum is artificially inoculated and/or the hazard of the Apolygus lucorum naturally occurs, and a plant that has an attenuated plant damage and/or has an increased plant yield compared with other plants that do not have the polynucleotide sequence encoding the ACh1 protein is harvested.

The “contact” in the present application means that insects and/or pests touch, stay and/or feed on a plant, a plant organ, a plant tissue or a plant cell, and the plant, plant organ, plant tissue or plant cell may be to express the insecticidal protein in vivo, or the plant, plant organ, plant tissue or plant cell has the insecticidal protein on the surface and/or has a microorganism that produces the insecticidal protein.

A term “control” and/or “prevention” in the present application means that the Apolygus lucorum is in contact with at least the ACh1 protein, and the growth of the Apolygus lucorum is inhibited and/or death is caused after the contact. Further, the Apolygus lucorum is in contact with at least the ACh1 protein by ingesting the plant tissue, and after the contact, all or part of the Apolygus lucorum is inhibited in growth and/or death is caused. The inhibition refers to sub-lethal, namely it is not lethal but may cause a certain effect in growth, behavior, physiology, biochemistry and tissue and other aspects, such as slow growth and/or stop. At the same time, the plant should be morphologically normal, and may be cultivated by a conventional method for consumption and/or generation of products. In addition, the plant and/or plant seed containing the polynucleotide sequence encoding the ACh1 protein for controlling the Apolygus lucorum, under the condition that the Apolygus lucorum is artificially inoculated and/or the Apolygus lucorum naturally occurs, has the reduced plant damage compared with non-transgenic wild plants, and the specific manifestations include, but are not limited to, improved stem resistance, and/or increased grain weight, and/or increased yield, and the like. The “control” and/or “prevention” effect of the ACM protein on the Apolygus lucorum may exist independently and may not be weakened and/or disappeared due to the presence of other substances that may “control” and/or “prevent” the Apolygus lucorum. Specifically, if any tissue of the transgenic plant (containing the polynucleotide sequence encoding the ACh1 protein) simultaneously and/or asynchronously exist with and/or produce the ACh1 protein and/or another substance that may control the Apolygus lucorum, the existence of the another substance neither affects the “control” and/or “prevention” effect of the ACh1 protein on the Apolygus lucorum, nor may cause the “control” and/or “prevention” effect to be completely and/or partially implemented by the another substance, which is independent of the ACh1 protein. Usually, in the field, the ingestion process of the plant tissue by the Apolygus lucorum is short and difficult to observe with naked eyes. Therefore, under the condition that the Apolygus lucorum is artificially inoculated and/or the Apolygus lucorum naturally occurs, for example, any tissues of the transgenic plant (containing the polynucleotide sequence encoding the ACh1 protein) have the dead Apolygus lucorum, and/or the Apolygus lucorum on which the growth is inhibited, and/or have the reduced plant damage compared with the non-transgenic wild plants, the method and/or the use of the present application is achieved. That is to say, the method and/or the use for controlling the Apolygus lucorum is achieved by allowing the Apolygus lucorum to be at least in contact with the ACh1 protein.

In the present application, the expression of the ACh1 protein in a transgenic plant may be accompanied by the expression of one or more Cry-like insecticidal proteins and/or Vip-like insecticidal proteins. Co-expression of such more than one insecticidal toxin in the same transgenic plant may be achieved by genetically engineering the plant to contain and express a desired gene. In addition, one plant (first parent) may express the ACM protein by a genetic engineering operation, and a second plant (second parent) may express the Cry-like insecticidal proteins and/or Vip-like insecticidal proteins by the genetic engineering operation. Offspring plants expressing all the genes introduced into the first and second parents are obtained by hybridizing the first and second parents.

RNA interference (RNAi) refers to a phenomenon that is highly conserved during the evolution process and induced by a double-stranded RNA (dsRNA), and a homologous mRNA is efficiently and specifically degraded. Therefore, an RNAi technology may be used in the present application to specifically knock out or shut down the expression of a specific gene in the target insect pest.

The Apolygus lucorum in the present application is a paurometabola insect belonging to Hemiptera Miridae. An adult has a body length of 5 mm and a width of 2.2 mm; and the adult is green and covered with short hair. The head of the adult is triangular and in yellow green, has protruding black compound eyes, and has no single eyes; there are 4 filamentous short antennae, and the antennae are about ⅔ of the body length; the length of the 2nd section equals a sum of lengths of the 3rd and 4th sections; the color of the antenna gradually darkens toward the end; and 1 section is in yellow green, and 4 sections are in black brown. The pronotum is in dark green and distributed with a plurality of small black dots; and the pronotum has a wide front edge. The scutellum is triangular and slightly protrudes; and the scutellum is in yellow green and has 1 shallow longitudinal grain at the central part. The forewing membranes are translucent and in dark grey, with remaining green. The feet are in yellow green; the color of the end of the intestinal segment and the financial segment are darker; the end of the hind foot section has brown ring spots; the hind foot section of the female is shorter than that of the male, which does not exceed the end of the abdomen; and there are 3 tarsi, and the ends of the tarsi are in black. An egg has a length of 1 mm, and is in yellow green and a long pocket shape. An egg cap is in cream yellow, has a depressed center and two protruding ends, and has no appendages on the edge. There are 5 nymphal instars, which is similar to the adults. The nymph is green at initial incubation and has pink compound eyes. The nymph is tawny at the 2nd instar; and wing buds appear at the 3rd instar, and exceed the 1st abdominal segment at the 4th instar. At the 2nd, 3rd and 4th instars, antenna ends and foot ends are in black brown. The nymph is in entire bright green after the 5th instar, and is densely covered with black fine hairs; the antennae are in faint yellow, with gradually darkened ends; and the eyes of the nymph are gray.

The ACh1 protein in the present application is a type of β-pore forming protein. The enzymatic cleavage activation in insect gut, receptor binding on the insect gut and a physicochemical environment in the insect gut are key points for achieving the effect of a β-pore forming protein. Only after the β-pore forming protein can be digested into active fragments and bound to the receptor on an epithelial cell membrane of the insect gut, it is possible to make a certain β-pore forming protein have an inhibitory activity against the pests. The receptor binding process requires accurate matching, and often a single amino acid difference in the pore forming protein or receptor protein can cause changes in binding to the same receptor. For example, after an aerolysin protein belonging to the same β-pore forming protein has qualitative changes in the virulence of a CTLL-2 cell line after R336A mutation (Osusky, Teschk et al, 2008). Likewise, since the receptor is changed, the virulence of the same β-pore forming protein may also be changed. For example, dsRNA is used to inhibit a HAVCR1 gene in a MDCK cell line, resulting in a hundred-fold difference in the virulence of an epsilon-toxin protein on cells (Ivie, Fennessey et al, 2011). The above fully indicates that the interaction between the β-pore forming protein and enzymes and receptors in insects is complex and unpredictable.

The genome of the plant, plant tissue or plant cell in the present application refers to any genetic materials in the plant, plant tissue or plant cell, and includes a cell nucleus and plastid and mitochondrial genome.

The polynucleotide and/or nucleotide described in the present application forms a complete “gene” that encodes a protein or polypeptide in the required host cell. It is very easily recognized by those skilled in the art that the polynucleotide and/or nucleotide of the present application may be placed under the control of a regulatory sequence in the target host.

It is well-known to those skilled in the art that DNA typically exists in a double-stranded form. In this arrangement, one strand is complementary to the other strand and vice versa. Other complementary strands of DNA are produced as a result of DNA replication in the plants. In this way, the present application includes the use of the polynucleotide exemplified in a sequence listing and complementary strands thereof. A “coding strand” as commonly used in the field refers to a strand that binds to an antisense strand. In order to express a protein in vivo, typically one strand of DNA is transcribed into a complementary strand of mRNA, it serves as a template for translation of the protein. mRNA is actually transcribed from the “antisense” strand of DNA. The “sense” or “coding” strand has a series of codons (the codon is three nucleotides, and a specific amino acid may be produced by reading three at a time), and it may be read as an open reading frame (ORF) to form a target protein or peptide. The present application also includes RNA that is functionally equivalent to the exemplified DNA.

The nucleic acid molecule or fragment thereof in the present application hybridizes to the ACh1 gene of the present application under stringent conditions. Any conventional nucleic acid hybridization or amplification methods may be used to identify the presence of the ACh1 gene of the present application. The nucleic acid molecule or fragment thereof is capable of specifically hybridizing with other nucleic acid molecules under certain circumstances. In the present application, if two nucleic acid molecules may form an anti-parallel double-stranded nucleic acid structure, it may be said that the two nucleic acid molecules may specifically hybridize with each other. If the two nucleic acid molecules show complete complementarity, one nucleic acid molecule is said to be a “complement” of the other nucleic acid molecule. In the present application, while each nucleotide of one nucleic acid molecule is complementary to the corresponding nucleotide of the other nucleic acid molecule, the two nucleic acid molecules are said to show the “complete complementarity”. If the two nucleic acid molecules may hybridize to each other with sufficient stability such that they anneal and bind to each other under at least conventional “low stringency” conditions, the two nucleic acid molecules are said to be “minimally complementary”. Similarly, if the two nucleic acid molecules may hybridize to each other with the sufficient stability such that they anneal and bind to each other under conventional “high stringency” conditions, the two nucleic acid molecules are said to have “complementarity”. Deviation from the complete complementarity is permissible as long as such deviation does not completely prevent the two molecules from forming the double-stranded structure. In order for a nucleic acid molecule to function as a primer or a probe, it only needs to be sufficiently complementary in its sequence, as to allow for the formation of the stable double-stranded structure under adopted particular solvent and salt concentration.

In the present application, the substantially homologous sequence is a section of a nucleic acid molecule, the nucleic acid molecule may specifically hybridize with a complementary strand of another matched nucleic acid molecule under highly stringent conditions. Suitable stringent conditions to promote the DNA hybridization, for example, treatment with 6.0× sodium chloride/sodium citrate (SSC) at about 45° C., and followed by washing with 2.0×SSC at 50° C., are well-known to those skilled in the art. For example, the salt concentration in a washing step may be selected from about 2.0×SSC and 50° C. under the low stringency conditions to about 0.2×SSC and 50° C. under the high stringency conditions. In addition, the temperature condition in the washing step may be increased from about 22° C. at a room temperature under the low stringency conditions to about 65° C. under the high stringency conditions. Both the temperature condition and the salt concentration may be changed, or one of which may be kept unchanged while the other variable is changed. Preferably, the stringency condition described in the present application may be specific hybridization in 6×SSC and 0.5% sodium dodecyl sulfate (SDS) solutions at 65° C., and then membrane-washing once with 2×SSC, 0.1% SDS and 1×SSC and 0.1% SDS.

Therefore, sequences that have the insecticidal activity and hybridize to SEQ ID NO:5, SEQ ID NO:6, SEQ ID NO:7, or SEQ ID NO:8 of the present application under the stringency condition are included in the present application. These sequences have at least about 40%-50% of the identity with the sequences of the present application, about 60%, 65% or 70% of the identity, even at least about 75%, 80%, 85%, 90%, 91%, 92%, 93%, 94%, 95%, 96%, 97%, 98%, 99% or greater sequence identity.

The genes and proteins described in the present application include not only a specific exemplified sequence, but also include parts and/or fragments (including internal and/or terminal deletion as compared with a full-length protein) that preserve the characteristics of the insecticidal activity of the specific exemplified protein, a variant, a mutant, a substitute (a protein with a substituted amino acid), a chimera and a fusion protein. The “variant” or “variation” refers to a nucleotide sequence encoding the same protein or encoding an equivalent protein with the insecticidal activity. The “equivalent protein” refers to a protein that has the same or substantially the same biological activity against the Apolygus lucorum as the claimed protein.

A “fragment” or “truncation” of the DNA molecule or protein sequence described in the present application refers to a portion of the original DNA or protein sequence (nucleotide or amino acid) involved or an artificially modified form thereof (for example, a sequence suitable for plant expression), the length of the aforementioned sequence may have a change, but is long enough to ensure that the (encoded) protein is an insect toxin.

A standard technology may be used to modify the gene and construct the genetic variant easily, for example, a technology for manufacturing a point mutation which is well-known in the field. As another example, U.S. Pat. No. 5,605,793 describes a method for producing additional molecular diversity using DNA reassembly after random fragmentation. The fragment of the full-length gene may be manufactured with a commercial endonuclease, and an exonuclease may be used according to a standard procedure. For example, enzymes such as Bal31 or site-directed mutagenesis may be used to systematically excise nucleotides from the ends of these genes. The genes encoding the active fragments may also be obtained with a plurality of restriction enzymes. The active fragments of these toxins may be obtained directly with proteases.

The present application may derive equivalent proteins and/or genes encoding these equivalent proteins from a β-pore forming protein isolate and/or a DNA library. There are various ways to obtain the insecticidal protein of the present application. For example, antibodies of the insecticidal protein disclosed and claimed in the present application may be used to identify and isolate other proteins from protein mixtures. In particular, the antibodies may arise from a portion of the protein that is most constant and most different from other β-pore forming proteins. These antibodies may then be used to specifically identify the equivalent proteins with the characteristic activity by immunoprecipitation, an enzyme-linked immunosorbent assay (ELISA), or a western blotting method. Antibodies of the proteins or the equivalent proteins or the fragments of such proteins disclosed in the present application may be easily prepared by the standard procedure in the field. The genes encoding these proteins may then be obtained from the microorganisms.

Due to the redundancy of genetic codons, many different DNA sequences may encode the same amino acid sequence. The generation of these alternative DNA sequences encoding the same or substantially same protein is within the technological level of those skilled in the art. These various DNA sequences are included within a scope of the present application. The “substantially same” sequence refers to a sequence with amino acid substitution, deletion, addition or insertion that does not substantially affect the insecticidal activity, and also includes a fragment that retains the insecticidal activity.

The substitution, deletion or addition of the amino acid sequence in the present application is a routine technology in the field, preferably such an amino acid change is: a small property change, namely conservative amino acid substitution that does not significantly affect the folding and/or activity of the protein; small deletion, typically deletion of about 1-30 amino acids; small amino- or carboxy-terminal extension, for example, amino-terminal extension of one methionine residue; and a small linker peptide, for example, the length of about 20-25 residues.

Examples of the conservative substitution are those that occur within the following amino acid groups: basic amino acids (such as an arginine, a lysine, and a histidine), acidic amino acids (such as a glutamic acid and an aspartic acid), polar amino acids (such as a glutamine, and an asparagine), hydrophobic amino acids (such as a leucine, an isoleucine, and a valine), aromatic amino acids (such as a phenylalanine, a tryptophan, and a tyrosine), and small molecular amino acids (such as a glycine, an alanine, a serine, a threonine, and a methionine). Those amino acid substitutions that generally do not change the specific activity are well-known in the field, and already described, for example, by N. Neurath and R. L. Hill in “Protein” published by Academic Press, New York in 1979. The most common interchanges are Ala/Ser, Val/Ile, Asp/Glu, Thu/Ser, Ala/Thr, Ser/Asn, AlaNal, Ser/Gly, Tyr/Phe, Ala/Pro, Lys/Arg, Asp/Asn, Leu/Ile, Leu/Val, Ala/Glu and Asp/Gly, and their opposite interchanges.

It is apparent to those skilled in the art that such substitutions may occur outside areas important to the function of the molecule, and still produce the active polypeptide. The amino acid residues that are essential for the activity of the polypeptide of the present application and are therefore selected not to be substituted may be identified according to methods known in the field, such as site-directed mutagenesis or alanine-scanning mutagenesis (referring to, for example, Cunningham and Wells, 1989, Science 244: 1081-1085). The latter technology is to introduce a mutation at each positively charged residue in the molecule, and to test the inhibitory activity of the mutant molecules obtained, thereby the amino acid residues that are important to the activity of the molecule are determined. Substrate-enzyme interaction sites may also be determined by analysis of its three-dimensional structure, this three-dimensional structure may be determined by technologies such as nuclear magnetic resonance analysis, crystallography, or photoaffinity labeling (referring to, for example, de Vos et al., 1992, Science 255:306-312; Smith et al., 1992, J. Mol. Biol 224:899-904; and Wodaver et al., 1992, FEBS Letters 309:59-64).

In the present application, the ACh1 protein includes, but is not limited to, SEQ ID NO:1, SEQ ID NO:2, SEQ ID NO:3, or SEQ ID NO:4, and amino acid sequences having certain identity with the amino acid sequence shown in SEQ ID NO:1, SEQ ID NO:2, SEQ ID NO:3, or SEQ ID NO:4 are also included in the present application. These sequences are typically greater than 78% of similarity/identity of the sequence of the present application, preferably greater than 85%, more preferably greater than 90%, even more preferably greater than 95%, and may be greater than 99%. Preferred polynucleotides and proteins of the present application may also be defined according to more specific ranges of the identity and/or similarity. For example, there are 78%, 79%, 80%, 81%, 82%, 83%, 84%, 85%, 86%, 87%, 88%, 89%, 90%, 91%, 92%, 93%, 94%, 95%, 96%, 97%, 98% or 99% of the identity and/or similarity with the sequences exemplified in the present application.

The regulatory sequence described in the present application includes, but is not limited to, a promoter, a transit peptide, a terminator, an enhancer, a leader sequence, an intron, and other regulatory sequences operably linked to the ACh1 protein.

The promoter is a promoter expressible in the plant, and the “promoter expressible in the plant” refers to a promoter that ensures the expression of the coding sequence linked to it in plant cells. The promoter expressible in the plant may be a constitutive promoter. Examples of the promoter that direct the constitutive expression in the plant include, but are not limited to, a 35S promoter derived from a cauliflower mosaic virus, a maize Ubi promoter, a promoter of a rice GOS2 gene and the like. Alternatively, the promoter expressible in the plant may be a tissue-specific promoter, namely the promoter directs the expression of the coding sequence to a higher level in some tissues of the plant, such as in a green tissue, than in other tissues of the plant (may be determined by a conventional RNA test), such as a PEP carboxylase promoter. Alternatively, the promoter expressible in the plant may be a wound-inducible promoter. The wound-inducible promoter or a promoter that directs a wound-induced expression pattern means that the expression of the coding sequence under the control of the promoter is significantly increased while the plant is subjected to a mechanical or insect-induced wound compared to normal growth conditions. Examples of the wound-inducible promoter include, but are not limited to, promoters of protease inhibitory genes (pin I and pin II) of potato and tomato and a promoter of a maize protease inhibitor gene (MPI). Examples of the wound-inducible promoter include, but are not limited to, promoters of protease inhibitory genes (pin I and pin II) of potato and tomato and a promoter of a maize protease inhibitor gene (MPI).

The transit peptide (also known as a secretion signal sequence or a targeting sequence) directs a transgenic product to a specific organelle or cellular compartment, the transit peptide may be heterologous to the receptor protein, for example, with a transit peptide sequence encoding a chloroplast to target the chloroplast, or using a ‘KDEL’ retention sequence to target an endoplasmic reticulum, or using CTPP of a barley lectin gene to target a vacuole.

The leader sequence includes, but is not limited to, a picornavirus leader sequence, such as an encephalomyocarditis virus 5′ non-coding region (EMCV) leader sequence; a potato Y virus group leader sequence, such as a maize dwarf mosaic virus (MDMV) leader sequence; a human immunoglobulin heavy chain binding protein (BiP); an untranslated leader sequence of coat protein mRNA of alfalfa mosaic virus (AMV RNA4); and a tobacco mosaic virus (TMV) leader sequence.

The enhancer includes, but is not limited to, a cauliflower mosaic virus (CaMV) enhancer, a figwort mosaic virus (FMV) enhancer, a carnation etched ring virus (CERV) enhancer, a cassava vein mosaic virus (CsVMV) enhancer, a mirabilis mosaic virus (MMV) enhancer, a cestrum yellow leaf curling virus (CmYLCV) enhancer, a cotton leaf curl multan virus (CLCuMV), a commellna yellow motile virus (CoYMV) and a peanut chlorella leaf strip virus (PCLSV) enhancer.

For monocot applications, the intron includes, but is not limited to, a maize hsp70 intron, a maize ubiquitin intron, an Adh intron 1, a sucrose synthase intron, or a rice Act1 intron. For dicot applications, the intron includes, but is not limited to, a CAT-1 intron, a pKANNIBAL intron, a PIV2 intron, and a “super ubiquitin” intron.

The terminator may be a suitable polyadenylation signal sequence functioning in the plant, including, but not limited to, a polyadenylation signal sequence derived from a nopaline synthase (NOS) gene of Agrobacterium tumefaciens, a polyadenylation signal sequence derived from a protease inhibitor II (pin II) gene, a polyadenylation signal sequence derived from a pea ssRUBISCO E9 gene, and a polyadenylation signal sequence derived from a α-tubulin gene.

The “operably linked” in the present application refers to association of nucleic acid sequences such that one sequence may provide a desired function for the linked sequence. In the present application, the “operably linked” may be to link a promoter with an interested sequence, so that the transcription of the interested sequence is controlled and regulated by the promoter. While the interested sequence encodes a protein and the expression of the protein is desired, the “operably linked” means that: the promoter is linked to the sequence, so that an obtained transcript is efficiently translated in a linkage mode. If the linkage of the promoter to the coding sequence is transcript fusion and the expression of the encoded protein is desired, such linkage is manufactured, so that the first translation initiation codon in the obtained transcript is an initiation codon of the coding sequence. Alternatively, if the linkage of the promoter to the coding sequence is translational fusion and the expression of the encoded protein is desired, such linkage is manufactured, so that the first translation initiation codon contained in a 5′ untranslated sequence is linked to the promoter, and a relationship between an obtained translation product and a translational open reading frame encoding the desired protein accords with the reading frame in the linkage mode. The nucleic acid sequence that may be “operably linked” includes, but is not limited to: sequences providing gene expression functions (namely gene expression elements such as a promoter, a 5′ untranslated region, an intron, a protein coding region, a 3′ untranslated region, a poly adenylation site and/or a transcription terminator), sequences providing DNA transfer and/or integration functions (namely a T-DNA border sequence, a site-specific recombinase recognition site, and an integrase recognition site), sequences providing selectivity functions (namely an antibiotic resistance marker, and a biosynthetic gene), sequences providing scoreable marker functions, sequences that assist in sequence operation in vitro or in vivo (namely a polylinker sequence, and a site-specific recombination sequence) and sequences providing replication functions (namely a bacterial replication origin, a autonomously replicating sequence, and a centromeric sequence).

In the present application, the “insecticide” or “insect resistance” means that it is toxic to crop pests, thereby the “control” and/or “prevention” of the crop pests is achieved. Preferably, the “insecticide” or “insect resistance” means that the crop pests are killed. More specifically, the target insect is the Apolygus lucorum.

The ACh1 protein in the present application is virulent to the Apolygus lucorum. The plant in the present application, especially the corn, the soybean and the cotton, contains an exogenous DNA in its genome. The exogenous DNA contains a nucleotide sequence encoding the ACh1 protein. The Apolygus lucorum is in contact with the protein by ingesting the plant tissue, and after the contact, the growth of the Apolygus lucorum is inhibited and/or death is caused. The inhibition means lethal or sub-lethal. At the same time, the plant should be morphologically normal, and may be cultivated under a conventional method for consumption and/or generation of products. In addition, the plant may substantially eliminate the need for a chemical or biological pesticide (the chemical or biological pesticide is an insecticide against the Apolygus lucorum targeted by the ACh1 protein).

The expression level of an insecticidal protein in the plant material may be detected by a plurality of methods described in the field, for example, by applying a specific primer to quantify mRNA encoding the insecticidal protein produced in the tissue, or directly specifically detecting the amount of the insecticidal protein produced.

Different tests may be applied to determine the insecticidal effect of the insecticidal protein in the plant. In the present application, the target insect is mainly the Apolygus lucorum.

In the present application, the ACh1 protein may have an amino acid sequence shown in SEQ ID NO:1, SEQ ID NO:2, SEQ ID NO:3, or SEQ ID NO:4 in a sequence listing. In addition to the coding region containing the ACh1 protein, other elements may also be included, such as a protein encoding a selectable marker.

In addition, an expression cassette containing the polynucleotide sequence encoding the ACh1 protein of the present application may also be expressed in the plant together with at least one protein encoding a herbicide resistance gene, the herbicide resistance gene includes, but not limited to, a glufosinate-ammonium resistance gene (such as a bar gene, and a pat gene), a Betanal resistance gene (such as a pmph gene), a glyphosate resistance gene (such as an EPSPS gene), a bromoxynil resistance gene, a sulfonylurea resistance gene, an anti-herbicide dalapon resistance gene, an anti-cyanamide resistance gene or a resistance gene of a glutamine synthase inhibitor (such as PPT), as to obtain the transgenic plant having both high insecticidal activity and herbicide resistance.

In the present application, the exogenous DNA is introduced into the plant, for example, the gene or expression cassette or recombinant vector encoding the ACh1 protein is introduced into the plant cell, and the conventional transformation method includes, but not limited to, agrobacterium-mediated transformation, micro-emission bombardment, direct DNA ingestion into a protoplast, electroporation, or whisker silicon-mediated DNA introduction.

The present application provides a use of an insecticidal protein and has the following advantages.

1. Prevention and treatment of internal causes: The prior art mainly controls the harm of the Apolygus lucorum by the external action namely the external causes, for example, the agricultural control, the chemical control, the physical control and the biological control; and the present application controls the Apolygus lucorum by producing the ACh1 protein that may kill the Apolygus lucorum in the plant, namely the Apolygus lucorum is controlled by the internal causes.

2. No pollution and no residue: Although the chemical control method used in the prior art plays a certain role in controlling the harm of the Apolygus lucorum, it also brings the pollution, damage and residue to humans, livestocks and farmland ecosystems; and using the method of the present application to control the Apolygus lucorum, the above adverse consequences may be eliminated.

3. Prevention and control during whole growth period: The methods used in the prior art to control the Apolygus lucorum are all staged, and the present application is to protect the plant during the whole growth period, and the transgenic plant (ACM protein) may be prevented from being attacked by the Apolygus lucorum from germination, growth, to flowering and fruiting.

4. Whole plant control: Most of the methods used in the prior art to control the Apolygus lucorum are localized, such as foliar spraying; and the present application protects the entire plant, for example, roots, leaves, stems, fruits, tassels, female ears, anthers or filaments of the transgenic plant (ACh1 protein) are all resistant to the attack of the Apolygus lucorum.

5. Stable effect: Whether it is the agricultural control method or the physical control method used in the prior art, it is necessary to use the environmental conditions to control the pests, and there are many variable factors; the present application is to express the ACh1 protein in the plant, which effectively overcomes the disadvantages of the unstable environmental conditions, and the control effect of the transgenic plant (ACh1 protein) of the present application is stable and consistent in different places, different times and different genetic backgrounds.

6. Simpleness, convenience and economy: The present application only needs to plant the transgenic plant capable of expressing the ACh1 protein, and does not need to adopt other measures, thereby reducing a lot of manpower, material resources and financial resources.

7. Complete effect: The methods used in the prior art to control the Apolygus lucorum are not thorough in effect, and only play a role in relieving; and the transgenic plant (ACh1 protein) of the present application may cause a large number of deaths of the larvae of the Apolygus lucorum.

The technical schemes of the present application are further described in detail below by drawings and embodiments.

### BRIEF DESCRIPTION OF THE DRAWINGS

FIG. 1 is a construction flowchart of a recombinant expression vector DBN01-P containing an ACh1 nucleotide sequence for the use of the insecticidal protein according to the present application.

### DETAILED DESCRIPTION OF THE EMBODIMENTS

The technical schemes of the use of the insecticidal protein of the present application are further described below by specific embodiments.

### Example 1: Acquisition and Synthesis of Gene

1. Acquisition of the Nucleotide Sequence

An amino acid sequence of an ACh1_1 insecticidal protein (309 amino acids) is shown in SEQ ID NO:1 in the sequence listing. An ACh1_1 nucleotide sequence (930 nucleotides) encoding the amino acid sequence corresponding to the ACh1_1 insecticidal protein in bacteria is shown in SEQ ID NO: 5 in the sequence listing. In the transgenic plant, an ACh1_1 plant nucleotide sequence (930 nucleotides) encoding the amino acid sequence corresponding to the ACh1_1 insecticidal protein is shown in SEQ ID NO:11 in the sequence listing.

An amino acid sequence of an ACh1_2 insecticidal protein (306 amino acids) is shown in SEQ ID NO:2 in the sequence listing. An ACh1_2 nucleotide sequence (921 nucleotides) encoding the amino acid sequence corresponding to the ACh1_2 insecticidal protein is shown in SEQ ID NO: 6 in the sequence listing. In the transgenic plant, an ACh1_2 plant nucleotide sequence (921 nucleotides) encoding the amino acid sequence corresponding to the ACh1_2 insecticidal protein is shown in SEQ ID NO:12 in the sequence listing.

An amino acid sequence of an ACh1_3 insecticidal protein (309 amino acids) is shown in SEQ ID NO:3 in a sequence listing. An ACh1_3 nucleotide sequence (930 nucleotides) encoding the amino acid sequence corresponding to the ACh1_3 insecticidal protein is shown in SEQ ID NO: 7 in the sequence listing. In the transgenic plant, an ACh1_3 plant nucleotide sequence (930 nucleotides) encoding the amino acid sequence corresponding to the ACh1_3 insecticidal protein is shown in SEQ ID NO:13 in the sequence listing.

An amino acid sequence of an ACh1_4 insecticidal protein (309 amino acids) is shown in SEQ ID NO:4 in a sequence listing. An ACh1_4 nucleotide sequence (930 nucleotides) encoding the amino acid sequence corresponding to the ACh1_4 insecticidal protein is shown in SEQ ID NO: 8 in the sequence listing. In the transgenic plant, an ACh1_4 plant nucleotide sequence (930 nucleotides) encoding the amino acid sequence corresponding to the ACh1_4 insecticidal protein is shown in SEQ ID NO:14 in the sequence listing.

2. Synthesis of Above Nucleotide Sequence

The ACh1_1 nucleotide sequence (as shown in SEQ ID NO:5 in the sequence listing), the ACh1_2 nucleotide sequence (as shown in SEQ ID NO:6 in the sequence listing), the ACh1_3 nucleotide sequence (as shown in SEQ ID NO:7 in the sequence listing) and the ACh1_4 nucleotide sequence (as shown in SEQ ID NO:8 in the sequence listing) are synthesized by Nanjing Genscript Biotechnology Co., Ltd.

### Example 2: Construction of Recombinant Expression Vector and Transformation of Recombinant Expression Vector into Escherichia coli to Obtain ACh1 Protein

1. Construction of Recombinant Expression Vector Containing ACh1 Gene

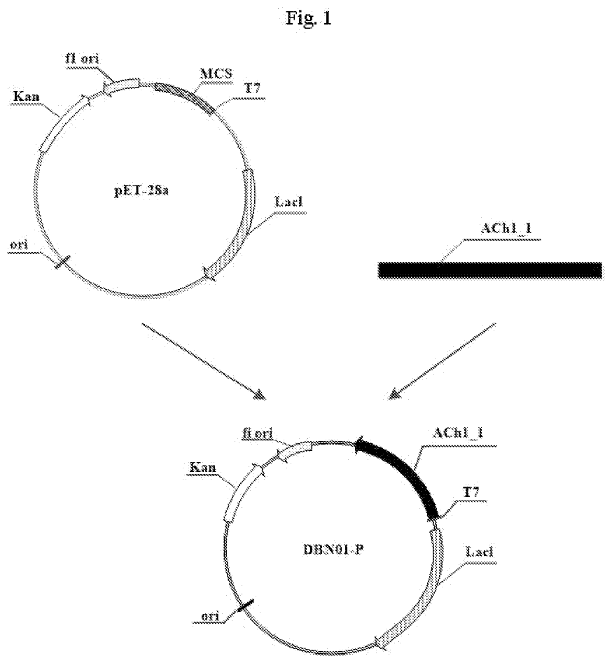

The synthesized ACh1_1 nucleotide sequence is linked into a protein expression vector pET28a (Novagen, USA, CAT: 69864-3); operation steps are performed according to the specification of the product pET28a vector of Novagen, so as to obtain a recombinant expression vector DBN01-P; and a construction flow is shown in FIG. 1 (where Kan represents a kanamycin resistance gene, f1 ori represents the origin of replication of phage f1, Lac1 is a Lac1 initiation codon, ACh1_1 is an ACh1_1 bacterial nucleotide sequence (SEQ ID NO:5), and MCS represents multiple cloning sites).

According to the above method for constructing the recombinant expression vector DBN01-P, the synthesized ACh1_2 nucleotide sequence is linked to the protein expression vector pET28a, so as to obtain a recombinant expression vector DBN02-P, and ACh1_2 is the ACh1_2 bacterial nucleotide sequence (SEQ ID NO:6).

According to the above method for constructing the recombinant expression vector DBN01-P, the synthesized ACh1_3 nucleotide sequence is linked to the protein expression vector pET28a, so as to obtain a recombinant expression vector DBN03-P, and ACh1_3 is the ACh1_3 bacterial nucleotide sequence (SEQ ID NO:7).

According to the above method for constructing the recombinant expression vector DBN01-P, the synthesized ACh1_4 nucleotide sequence is linked to the protein expression vector pET28a, so as to obtain a recombinant expression vector DBN04-P, and ACh1_4 is the ACh1_4 bacterial nucleotide sequence (SEQ ID NO:8).

2. Transformation of Recombinant Expression Vector into Escherichia coli to Obtain ACh1 Protein

Then, the recombinant expression vectors DBN01-P, DBN02-P, DBN03-P, and DBN04-P are transformed into Escherichia coli BL21(DE3) competent cells (Transgen, China, CAT: CD501) by a heat shock method; a positive colony is picked and placed in an LB liquid medium (10 g/L of a tryptone, 5 g/L of a yeast extract, 10 g/L of NaCl, 100 mg/L of an ampicillin, and pH is adjusted to 7.5 with NaOH); and culture is performed for 16 h at 37° C. and at 200 r/min. The culture solution is then transferred to an YT culture medium according to the proportion of 1:10; and culture is performed at 37° C. and at 200 r/min. When an OD=600 value of the culture solution reaches 0.6-0.8, IPTG is added until a final concentration is 0.5 mM, so as to perform inducible expression for 6 h, and the culture solution is centrifuged to collect the cells; the supernatant is discarded, resuspending is performed after PBS is added, and then ultrasonic disruption is performed; and the expression protein is detected by SDS-PAGE, the protein concentration is estimated, and preservation is performed at −20° C. for later use.

### Example 3, Identification of Inhibitory Activity Against Apolygus lucorum by Feeding ACh1 Protein

Inhibitory activity against the Apolygus lucorum is detected with the ACh1_1, ACh1_2, ACh1_3, and ACh1_4 proteins obtained in 2 in the Example 2. A total of 4 treatments are designed for each pest, which respectively are ACh1_1, ACh1_2, ACh1_3, and ACh1_4; and 1 negative control treatment is designed, which is GFP. Protein liquid of ACh1_1, ACh1_2, ACh1_3, ACh1_4, and GFP are respectively mixed in feed, and a final concentration is 500 μg/g. Each group of treatments is repeated for 6 times.

TABLE 1
Anti-insect results of Apolygus lucorum fed with ACh1 protein

| Serial number | Test insect |
|---|---|
| of proteins | Apolygus lucorum |
| ACh1_1 | + |
| ACh1_2 | + |
| ACh1_3 | + |
| ACh1_4 | + |
| GFP | − |

“+” means that there is inhibitory activity against the pest; and “−” means that there is no inhibitory activity against the pest.

Results of Table 1 show that, the ACh1_1, ACh1_2, ACh1_3, and ACh1_4 proteins have desirable inhibitory activity against the Apolygus lucorum.

Therefore, it indicates that the ACh1_1, ACh1_2, ACh1_3, and ACh1_4 proteins show resistance activity against the Apolygus lucorum, and this activity is sufficient to have adverse effects on the growth of the Apolygus lucorum, so that the Apolygus lucorum can be controlled in the fields. In addition, it is also possible to reduce the occurrence of diseases on the transgenic ACh1 plants by controlling the damage of the Apolygus lucorum, thereby greatly improving the yield and quality of the transgenic ACh1 plants.

In conclusion, through the use of the insecticidal protein of the present application, ACh1 protein that can kill the Apolygus lucorum is produced in bacteria and/or a plant body to control the Apolygus lucorum. Compared with an agricultural control method, a chemical control method and a physical control method used in the prior art, the present application achieves the protection of whole growth period and whole plant on the plants so as to control the infestation of the Apolygus lucorum, and is pollution-free, residue-free, stable in effect, thorough, simple, convenient and economical.

Finally, it should be noted that the above embodiments are only used to illustrate the technical schemes of the present application and not to limit them. Although the present application is described in detail with reference to the preferred embodiments, those of ordinary skill in the art should understand that the technical schemes of the present application may be modified or equivalently replaced without departing from the spirit and scope of the technical schemes of the present application.

What is claimed is:

1. A method for controlling Apolygus lucorum, comprising allowing the Apolygus lucorum to be at least in contact with an ACh1 protein;
preferably, the ACh1 protein is present in a host cell that produces at least the ACh1 protein, and the Apolygus lucorum is in contact with at least the ACh1 protein by ingesting the host cell; and
more preferably, the ACh1 protein is present in bacteria or a transgenic plant that produces at least the ACh1 protein, the Apolygus lucorum is in contact with at least the ACh1 protein by ingesting the bacterium or tissue of the transgenic plant, and after contacting, the growth of the Apolygus lucorum is inhibited and/or death is caused, so as to achieve the control of the damage of the Apolygus lucorum to plants.

2. The method for controlling Apolygus lucorum according to claim 1, wherein the transgenic plant is cotton, soybean, or rape.

3. The method for controlling Apolygus lucorum according to claim 1, wherein the tissue of the transgenic plant is a bud, a leaf, a cotton boll, a tassel, an ear, or a filament.

4. The method for controlling Apolygus lucorum according to claim 1, wherein the ACh1 protein is an ACh1_1 protein, an ACh1_2 protein, an ACh1_3 protein, or an ACh1_4 protein.

5. The method for controlling Apolygus lucorum according to claim 4, wherein the ACh1 protein has an amino acid sequence shown in SEQ ID NO:1, SEQ ID NO:2, SEQ ID NO:3, or SEQ ID NO:4.

6. The method for controlling Apolygus lucorum according to claim 4, wherein the ACh1 protein has a nucleotide sequence in the bacteria that is shown in SEQ ID NO:5, SEQ ID NO:6, SEQ ID NO:7, or SEQ ID NO:8; and the ACh1 protein has a nucleotide sequence in the transgenic plant that is shown in SEQ ID NO:11, SEQ ID NO:12, SEQ ID NO:13, or SEQ ID NO:14.

7. The method for controlling Apolygus lucorum according to claim 1, wherein the transgenic plant further comprises at least one second nucleotide different from the nucleotide encoding the ACh1 protein.

8. The method for controlling Apolygus lucorum according to claim 7, wherein the second nucleotide encodes a Cry-like insecticidal protein, a Vip-like insecticidal protein, a protease inhibitor, lectin, α-amylase, or a peroxidase.

9. The method for controlling Apolygus lucorum according to claim 7, wherein the second nucleotide is a dsRNA that inhibits an important gene in a target insect pest.

10. A method of producing a plant for controlling Apolygus lucorum, comprising introducing a polynucleotide sequence encoding an ACh1 protein into a genome of the plant.

11. The method of producing a plant for controlling Apolygus lucorum according to claim 10, wherein the polynucleotide sequence of the ACh1 protein is shown in SEQ ID NO:11, SEQ ID NO:12, SEQ ID NO:13 or SEQ ID NO:14.

12. The method of producing a plant for controlling Apolygus lucorum according to claim 10, wherein the ACh1 protein has an amino acid sequence shown in SEQ ID NO:1, SEQ ID NO:2, SEQ ID NO:3, or SEQ ID NO:4.

13. A method for producing a plant seed for controlling Apolygus lucorum, comprising hybridizing a plant obtained by the method according to claim 10 with a second plant, so as to produce a seed containing a polynucleotide sequence encoding an ACh1 protein.

14. A method for cultivating a plant for controlling Apolygus lucorum, comprising:
planting at least one plant seed, wherein a genome of the plant seed comprises a polynucleotide sequence encoding an ACh1 protein;
growing the plant seed into a plant; and
growing the plant under conditions that the Apolygus lucorum is artificially inoculated and/or the hazard of the Apolygus lucorum naturally occurs, and harvesting a plant that has an attenuated plant damage and/or has an increased plant yield compared with other plants that do not have the polynucleotide sequence encoding the ACh1 protein.

15. The method of cultivating a plant for controlling Apolygus lucorum according to claim 14, wherein the polynucleotide sequence of the ACh1 protein is shown in SEQ ID NO:11, SEQ ID NO:12, SEQ ID NO:13 or SEQ ID NO:14.

16. The method of cultivating a plant for controlling Apolygus lucorum according to claim 14, wherein the ACh1 protein has an amino acid sequence shown in SEQ ID NO:1, SEQ ID NO:2, SEQ ID NO:3, or SEQ ID NO:4.


SEQUENCE LISTING (14 sequences)

SEQ ID NO: 1 - protein, 309 aa, synthetic construct
MIFLAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 2 - protein, 306 aa, synthetic construct
MAILDLKSLVLNAINYWGPKNNNGIQGGDFGYPISEKQIDTSIITSTHPRLIPHDLTIPQNLETIFTTTQVLTNNTDLQQSQTVSFAKKTTTTTATSTTNGWTEGGKISDTLEEKVSVSIPFIGEGGGKNSTTIEANFAHNSSTTTSQEASTDIEWNISQPVLVPPRKQVVATLVIMGGNFTIPMDLMTTIDSTEHYSGYPILTWISSPDNSYSGRFMSWYFANWPNLPSGFGPLNSDNTVTYTGSVVSQVSAGVYATVRFDQYDIHNLWTIEKTWYARHATLHNGKKISINNVTEMAPTSPIKTN

SEQ ID NO: 3 - protein, 309 aa, synthetic construct
MAILDLKSLVLNAINYWGPKNNNGIQGGDFGYPISEKQIDTSIITFTHPRLIPYDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPSKQVVATLVIMGGNFTIPMDLMTTIDSTEHYSHYSGYPILTWISSPDNSYSGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 4 - protein, 309 aa, synthetic construct
MIFLAILDLKSLVLNAINYWGPKNNNGIQGGDFGYPISEAQIDTSIITSTHPRLIPHDLTIPQNLETIFTTTQVLTNNTDLQQSQTVSFAKKTTTTTSTSTTNGWTEGGAISDTLEEKVSVSIPFIGEGGGKNSTTIEANFAHNSSTTTFQQASTDIEWNISQPVLVPPAKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLATIEKTWYARHATLHNGKKISINNVTEMAPTSPIKTN

SEQ ID NO: 5 - DNA, 930 bp, synthetic construct
  source 1..930: mol_type = other DNA
atgatttttttggctatactagatttaaaatcactggtgttgaatgcgatcaactattggggtccgaaaaacaacaacggtattcagggtggtgattttggttacccgattagcgaaaagcagattgacaccagcattattacctctacccacccgcgtctgattccgcatgatcttacgatcccgcagaatttggaaaccatttttaccactacgcaagtcctgacgaacaacaccgacctgcagcaaagccaaaccgttagctttgctaaaaagaccactaccacgacctcgacctctacgacgaacggctggaccgagggcggcaagatctccgataccttggaggaaaaggtgtccgtgtctatcccgttcatcggcgaaggcggcggtaagaactccaccacaatcgaggcgaatttcgcgcataactcctcaacgaccacatttcaacaggcaagcaccgacatcgagtggaatatttcccaaccggttctggtgccgccgcgcaaacaggttgttgcgaccttggttattatgggtggcaatttcaccatcccgatggatttaatgaccacgatcgacagcactgagcactacagcggttacccgatcctgacctggattagcagcccggataactcctacaatggcccattcatgagctggtacttcgcgaactggccgaatctgccgagcggtttcggcccactgaacagcgacaacacggttacctataccggtagcgttgtctcgcaagtgagcgcgggggtgtatgccacggtgcgttttgatcagtatgacatccacaatctgcgcaccatcgagaagacctggtacgcccgtcatgctaccctgcacaacggtaaaaaaatcagcatcaataacgtgaccgaaatggcaccgacctctccgatcaaaaccaactaa

SEQ ID NO: 6 - DNA, 921 bp, synthetic construct
  source 1..921: mol_type = other DNA
atggctatcctggacctcaagtcgctggtgctcaacgccattaactactgggggccaaagaacaacaacggcattcaaggcggggacttcggctaccctatcagcgagaagcagattgataccagcatcattacctccacccacccgaggctcatcccacatgacctgacaattccccagaacctcgaaactatcttcaccacgacacaagttcttacaaacaatactgatctccagcaatctcagaccgtgtcatttgctaagaagactaccacgacaactgcgacctccaccacgaacggctggacggagggcgggaagatctctgacacgctggaggaaaaggtcagcgtttcgatcccgttcattggcgagggcggcggcaagaactccacaactatcgaagccaacttcgcgcacaattccagcaccacgacatcacaggaggctagcaccgatatcgagtggaacatttcgcagccagtgctcgtcccgccacggaagcaagtggtcgcaacactcgtgatcatgggcggcaatttcactattccgatggacctgatgactaccatcgattccaccgaacattacagcgggtatccaatccttacgtggattagctcaccggacaactcgtactctggccggttcatgtcttggtattttgcgaactggcctaatctgccctcagggttcggccctcttaactccgataatactgtgacctacacggggtctgttgtgtcacaggtttccgccggcgtgtacgcgacagtcagatttgaccaatatgatatccacaacctttggaccattgagaagacgtggtatgctaggcacgcaactctccataatggcaagaagattagcatcaacaatgtgacagaaatggctccaacgtcccctatcaagacaaactga

SEQ ID NO: 7 - DNA, 930 bp, synthetic construct
  source 1..930: mol_type = other DNA
atggccattctggatctgaagagcctggttctgaacgcgattaactactgggggccgaaaaataacaacggcattcagggtggcgacttcgggtacccgatctcagagaaacagatcgacacttccatcattacctttacgcacccgcgcctgattccatatgatctgacgatcccgcagaacctggaaaccatcttcacgacgactcaagttctgaccaacaacaccgatctgcagcagtcgcaaaccgtgtccttcgcaaaaaagacaacaaccacgactagcaccagcacaaccaacggctggaccgaaggggggaaaatttccgataccctcgaggagaaagtgagcgttagtatcccgtttatcggagaaggtggtggcaaaaattccaccaccattgaagccaatttcgcgcacaatagctcaaccaccaccttccaacaggccagcactgatatcgaatggaacattagccaaccggtgcttgtacctccctcgaaacaggtggtggccaccttagtcattatgggcggcaacttcacaattcctatggatctgatgaccactatcgacagtaccgaacactacagccattattcgggctatcccatcttaacttggatttcctccccggataatagctactccggtccgtttatgtcctggtatttcgctaattggcctaacctgccgagtgggtttggtccgttaaattccgacaataccgtgacctatacgggcagcgtggtatcacaagtatcggcaggcgtttatgcgaccgtccgtttcgatcaatatgacattcacaacctgcgtacgatcgagaaaacctggtacgcccgccacgccacgctgcataacggcaaaaaaattagcattaacaacgttaccgaaatggcgccgacctcgccgatcaaaaccaactga

SEQ ID NO: 8 - DNA, 930 bp, synthetic construct
  source 1..930: mol_type = other DNA
atgatttttttggctatactagatttaaaatccctggttttgaatgcgatcaactactggggtccgaagaacaacaacggcatccaaggtggtgatttcggttatccgatcagcgaagcccagattgacacctcaatcatcacctccacccatccgcgcctgattccgcacgatttgaccatcccgcaaaacttggaaaccatctttaccacgacccaggtactgaccaacaacaccgacctgcaacagagccaaaccgtgtcttttgctaaaaagactaccacgacgacctctacctctacaacgaacggctggaccgagggtggcgcaatctccgacaccctggaggaaaaggtgagcgtttctatcccgttcattggtgaaggcggcggaaaaaacagcactacgattgaagcgaacttcgcgcacaacagcagcactacgacctttcagcaagcgagcaccgatattgagtggaatatttcccagccggttctggtgccgcctgcaaagcaggtcgtggccactctggttattatgggtggtaatttcaccataccgatggatttaatgaccacgattgatagcaccgagcactacagcggctacccgatcctgacctggattagcagcccggataatagttataatggtccgttcatgagctggtacttcgccaactggccaaatcttccgagcggctttggcccactgaattctgacaacaccgtgacctataccggtagcgtggtctcgcaagtgagcgcgggtgtttatgcgaccgttcgttttgaccagtacgacatccacaatctcgcgaccatcgagaaaacctggtacgctcgtcatgctaccctgcataacggcaaaaagatcagcattaataacgttacggagatggcaccgacctcaccgattaaaaccaactaa

SEQ ID NO: 9 - protein, 653 aa, synthetic construct
MANPNNRSEHDTIKVTPNSELQTNHNQYPLADNPNSTLEELNYKEFLRMTEDSSTEVLDNSTVKDAVGTGISVVGQILGVVGVPFAGALTSFYQSFLNTIWPSDADPWKAFMAQVEVLIDKKIEEYAKSKALAELQGLQNNFEDYVNALNSWKKTPLSLRSKRSQDRIRELFSQAESHFRNSMPSFAVSKFEVLFLPTYAQAANTHLLLLKDAQVFGEEWGYSSEDVAEFYRRQLKLTQQYTDHCVNWYNVGLNGLRGSTYDAWVKFNRFRREMTLTVLDLIVLFPFYDIRLYSKGVKTELTRDIFTDPIFLLTTLQKYGPTFLSIENSIRKPHLFDYLQGIEFHTRLRPGYFGKDSFNYWSGNYVETRPSIGSSKTITSPFYGDKSTEPVQKLSFDGQKVYRTIANTDVAAWPNGKVYLGVTKVDFSQYDDQKNETSTQTYDSKRNNGHVSAQDSIDQLPPETTDEPLEKAYSHQLNYAECFLMQDRRGTIPFFTWTHRSVDFFNTIDAEKITQLPVVKAYALSSGASIIEGPGFTGGNLLFLKESSNSIAKFKVTLNSAALLQRYRVRIRYASTTNLRLFVQNSNNDFLVIYINKTMNKDDDLTYQTFDLATTNSNMGFSGDKNELIIGAESFVSNEKIYIDKIEFIPVQL

SEQ ID NO: 10 - DNA, 1962 bp, synthetic construct
  source 1..1962: mol_type = other DNA
atggcgaaccccaacaataggtccgagcacgacacgatcaaggtcacacccaactccgagctgcagactaatcacaatcagtacccactcgccgacaaccccaatagcacgctcgaggagctgaactacaaggagttcctccgcatgacggaggactcctccacagaggtgctggataattctactgtcaaggacgcggttggcaccgggatctcagtggtcgggcagattctcggcgttgtgggggtcccattcgctggcgctctgacgtccttctaccagagcttcctcaacacaatctggccatccgatgccgacccttggaaggccttcatggcgcaggtggaggtcctgatcgacaagaagattgaggagtacgccaagtcgaaggctctcgctgagctgcagggcctccagaacaatttcgaggactacgtgaacgcgctcaattcatggaagaagaccccactctccctgaggagcaagcgctcgcaggaccgcatcagggagctgttctcgcaggcggagtctcacttcaggaactctatgccctcattcgctgtgagcaagttcgaggtcctcttcctgccaacgtacgcccaggccgcgaacacacatctcctgctcctgaaggacgctcaggtgttcggggaggagtggggctactcgtctgaggatgtcgccgagttctaccgcaggcagctcaagctgacgcagcagtacacagaccactgcgttaactggtacaatgtgggcctgaacgggctcaggggctctacgtacgatgcgtgggtgaagttcaatcggttccggcgcgagatgaccctgacggtcctcgatctgatcgttctcttccccttctacgacattcgcctgtactcgaagggcgtcaagacggagctcacaagggatatcttcactgacccaattttcctcctgaccacgctccagaagtacggccctaccttcctgtcaatcgagaactccattcgcaagccgcacctgttcgactacctccagggcatcgagttccatacccggctgcgccccgggtacttcggcaaggattctttcaactactggtcagggaattacgtggagacacgcccgtcaatcggctcatccaagacaattacttcaccgttctacggggataagtccacagagcccgtccagaagctctccttcgacggccagaaggtttaccgcactatcgccaacaccgatgtggctgcctggccgaatgggaaggtctacctgggcgttacaaaggtggacttcagccagtacgacgatcagaagaacgagacatccacgcagacatacgattctaagaggaacaatggccatgtgagcgcgcaggattcgatcgaccagctcccgcccgagacaactgacgagcctctggagaaggcttacagccaccagctgaactacgccgagtgcttcctcatgcaggacaggcggggcaccatcccgttcttcacttggacccatcggtcggtcgatttcttcaacactatcgacgctgagaagattacccagctccctgtcgttaaggcgtacgctctgagctcgggcgcttccatcattgagggcccagggttcactggcgggaacctcctgttcctcaaggagtcttcaaatagcatcgctaagttcaaggttacgctgaactcggctgctctcctgcagaggtaccgggtccgcattcgctacgcgtccaccacgaacctcaggctgttcgttcagaatagcaacaatgacttcctcgtgatctacattaacaagaccatgaataaggacgatgacctgacttaccagaccttcgacctcgcgacaactaactccaatatggggttcagcggcgataagaacgagctgatcattggcgctgagtccttcgtgtccaacgagaagatctacattgataagattgagttcattcccgtccagctctga

SEQ ID NO: 11 - DNA, 930 bp, synthetic construct
  source 1..930: mol_type = other DNA
atgattttcctcgctatcctggacctcaagtcgctggtgctcaacgccattaactactgggggccaaagaacaacaacggcattcaaggcggggacttcggctaccctatcagcgagaagcagattgataccagcatcattacctccacccacccgaggctcatcccacatgacctgacaattccccagaacctcgaaactatcttcaccacgacacaagttcttacaaacaatactgatctccagcaatctcagaccgtgtcatttgctaagaagactaccacgacaacttcaacctccaccacgaacggctggacggagggcgggaagatctctgacacgctggaggaaaaggtcagcgtttcgatcccgttcattggcgagggcggcggcaagaactccacaactatcgaagccaacttcgcgcacaattccagcaccacgacatttcagcaagctagcaccgatatcgagtggaacatttcgcagccagtgctcgtcccgccacggaagcaagtggtcgcaacactcgtgatcatgggcggcaatttcactattccgatggacctgatgactaccatcgattccaccgaacattacagcgggtatccaatccttacgtggatttcgtctcctgacaactcgtacaatggccccttcatgtcttggtattttgcgaactggcctaatctgccctcagggttcggccctcttaactccgataatactgtgacctacacggggtctgttgtgtcacaggtttccgccggcgtgtacgcgacagtcagatttgaccaatatgatatccacaaccttcgcaccattgagaagacgtggtatgctaggcacgcaactctccataatggcaagaagattagcatcaacaatgtgacagaaatggctccaacgtcccctatcaagacaaactga

SEQ ID NO: 12 - DNA, 921 bp, synthetic construct
  source 1..921: mol_type = other DNA
atggctatccttgatcttaagtcccttgtgctaaatgctatcaattattggggacctaaaaataataatggcattcaaggaggcgattttggatacccgatttcggaaaaacaaattgacacttctattattacatctactcatccgcgtctcattcctcacgatcttacaattccacaaaacttagaaacaatttttactaccactcaagttttaacgaataatacagatttgcaacaatcacaaacggtttcgtttgcgaagaagacaacaactaccacagcgacttccaccacaaacggttggactgaagggggaaaaatttctgacactttagaagagaaggtttcagtctcgatcccttttataggagaagggggtggaaagaactcaacgacaatagaagcaaacttcgcacacaactcttcgactactacatcccaggaagctagcacagatatagaatggaacatctcacagcctgtgcttgtgccgccaagaaaacaagttgttgccactttggtcataatgggaggtaacttcactatcccaatggatctaatgacaacaattgattctaccgaacactattccggttacccgatattaacctggatatcaagtccagataattcatactctggaagattcatgtcatggtatttcgctaactggcctaatcttccttctggattcggacctttaaactctgataatacggtgacatatactggaagcgtggtgtctcaagtgagcgctggtgtgtatgcaacggtcagattcgaccagtacgatatacacaatttatggacgattgaaaagacatggtatgctagacatgctactctccacaacggaaagaaaatcagcattaataacgttaccgaaatggctccaactagtcctatcaaaactaactga

SEQ ID NO: 13 - DNA, 930 bp, synthetic construct
  source 1..930: mol_type = other DNA
atggcaattctagatttaaagagcttagtcttaaatgccataaactactggggaccgaaaaataacaatgggattcaaggaggtgatttcggatacccaatttctgagaagcaaatcgatacatctattataacatttactcatccaagacttatcccgtacgatttaaccatccctcaaaacctcgaaactatctttactacgacacaggttttgacaaacaacaccgatcttcagcagagccagacagttagtttcgccaagaagactactactaccacctctacttcaaccactaacggatggaccgagggtgggaagatttccgatactcttgaggaaaaagtgagcgttagcataccgtttatcggagaaggtggaggaaagaattctacgactatcgaggctaatttcgctcacaactcttcgactacgacttttcaacaagcatctactgatattgagtggaatatttctcagcctgtattggttccaccttcaaaacaagttgtggctacattagtcatcatggggggtaactttacgattcctatggacctgatgacaacaattgattcaactgaacattactcgcattattcaggttacccaatcctcacatggatttcaagcccagacaacagctattcgggaccatttatgtcatggtacttcgcaaattggcccaatctgccgagtggatttggtcctctcaattcggataacacagttacatacactggttcggttgttagtcaagtaagtgctggagtgtatgcaaccgtcagattcgatcagtatgatattcataatcttaggactatcgagaaaacatggtatgctagacacgcgacgcttcataatggaaaaaagatttccattaacaatgtgactgagatggcaccaacgtctccgattaaaactaattga

SEQ ID NO: 14 - DNA, 930 bp, synthetic construct
  source 1..930: mol_type = other DNA
atgatcttcctcgcgattttggatctaaagtcacttgtactcaatgcgattaattattggggacctaaaaacaataatggcattcaaggaggagatttcggttacccaatatctgaagctcagatcgatacatccattattaccagcactcatcctaggctcattcctcatgaccttactattccacagaaccttgaaactattttcacaacgacgcaagtgttgacaaataacaccgacttgcaacaatctcagactgtttcttttgctaagaaaaccactacaacaacatctacttcaaccacgaatggatggacggaaggaggtgctatatccgacaccttggaagaaaaggtgtcggtgtcaatccctttcattggagaaggcggtgggaagaactctactacaattgaagctaactttgctcataattcttcgactacaacttttcagcaagcttccacggatatcgaatggaacatttcacagcctgttttggtgccacccgcaaaacaggttgtggcaactttggtgattatgggtgggaattttactatccctatggatcttatgacgactattgattctactgagcactacagcggatatcctattcttacctggatctcttcacctgacaatagctataacgggccttttatgagttggtattttgcaaattggcccaacctcccaagtggattcgggccacttaactctgataatacagttacctacactggttccgtggttagtcaagtgtcggctggtgtgtatgctaccgtgcgttttgatcaatatgatattcataacctcgctacaattgaaaagacttggtacgctcgacacgcaacgctccacaatggtaaaaaaatctcgattaacaatgttaccgagatggcacccactagtccaatcaagactaactga